(19) United States
(12) Patent Application Publication
(10) Pub. No.: US 2026/0162267 A1
(43) Pub. Date: Jun. 11, 2026

(54) Methods and Systems of Generating Perfusion Parametric Maps

(71) Applicant: The Board of Trustees of the Leland Stanford Junior University, Stanford, CA (US)

(72) Inventors: Elizabeth Tong, San Francisco, CA (US); Ettore Biondi, Los Angeles, CA (US); Guillaume Barnier, Stanford, CA (US); Gregory Forbes, Stanford, CA (US); Aaron Sossin, Stanford, CA (US); Vivian Zhu, Stanford, CA (US); Siyu Gai, Berkeley, CA (US); Salmonn Talebi, Albany, CA (US)

(73) Assignee: The Board of Trustees of the Leland Stanford Junior University, Stanford, CA (US)

(21) Appl. No.: 18/708,432

(22) PCT Filed: Nov. 8, 2022
(86) PCT No.: PCT/US2022/079472

§ 371 Date: May 8, 2024

Related U.S. Application Data
(60) Provisional application No. 63/263,750, filed on Nov. 8, 2021.

(51) Int. Cl. G06T 7/00; A61B 5/00; A61B 5/055; A61B 6/03; G01R 33/563; G06T 5/60; G06T 5/70; G16H 50/20

(52) U.S. Cl. CPC G06T 7/0016; A61B 5/0042; A61B 5/055; A61B 5/4064; A61B 6/03; G01R 33/56366; G06T 5/60; G06T 5/70; G16H 50/20; G06T 2207/10081; G06T 2207/10088; G06T 2207/10104; G06T 2207/10121; G06T 2207/10132; G06T 2207/20084; G06T 2207/30016; G06T 2207/30104

(57) ABSTRACT

Methods and systems for generating parametric perfusion maps are provided. A computational learning model can be utilized to extract temporal or spatial features of perfusion images. Extracted temporal and spatial features can be utilized to generate parametric perfusion maps.

### CROSS REFERENCE TO RELATED APPLICATIONS

The current application is a national stage of PCT Patent Application No. PCT/US2022/079472 entitled “Methods and Systems of Generating Perfusion Parametric Maps” to Tong et al., filed Nov. 8, 2022, which claims the benefit of U.S. Provisional Patent Application No. 63/263,750, entitled “Methods and Systems of Generating Perfusion Parametric Maps” to Tong et al., filed Nov. 8, 2021, the disclosures of which are incorporated herein by reference in their entirety.

### TECHNOLOGICAL FIELD

The disclosure is generally directed to methods and systems to generate perfusion parametric maps and applications thereof, including detecting and assessing disorders with cerebral perfusion abnormalities, including (but not limited to) cerebrovascular disorder (such as stroke, vascular malformations, transient-ischemic attacks), hypo-perfusion disorders (such as post-ictal phase of seizure, post-radiation necrosis), and hyper-perfusion disorders (such as brain malignancy including brain tumor or brain metastases, ictal phase of seizure, inflammation, and infections).

### BACKGROUND

Perfusion is the passage of fluid through an organ or tissue by way of blood vessels. Perfusion mapping utilizes an imaging modality to observe, record, and quantify perfusion. Perfusion maps measure blood flow to and blood uptake by the brain, and are especially useful for detecting and diagnosing disorders of the brain in which blood flow or blood uptake is disturbed, including cerebrovascular disorder (such as stroke, vascular malformations, transient-ischemic attacks), hypo-perfusion disorders (such as post-ictal phase of seizure, post-radiation necrosis), and hyper-perfusion disorders (such as brain malignancy including (such as brain tumor or brain metastases, ictal phase of seizures, inflammation, and infections).

Stroke is a medical condition in which poor blood flow within the brain causes damage, resulting in loss of brain cells and potentially brain function. There are two main types of stroke: ischemic and hemorrhagic. Ischemic stroke is the loss of blood flow, typically due to an obstruction in a blood vessel. Hemorrhagic stroke is an internal bleeding within the skull (typically within the brain tissue). Each type can result in an inadequate amount of oxygen reaching various portions of brain tissue, causing brain cell loss and damage. Loss of oxygen can lead to some brain tissue being infarcted (often referred to the core of the stroke lesion) and is typically unsalvageable. Further, the tissue surrounding the core (often referred to the penumbra of the stroke lesion) is damaged but potentially salvageable. Quick identification of the core and the penumbra can help medical personnel diagnose and treat stroke patients efficiently and effectively.

### SUMMARY

Several embodiments are directed to methods and systems of generating perfusion parametric maps. In many embodiments, scans of perfusion images are acquired over a period time. In several embodiments, time-intensity curves are generated for one or more voxels of the scans of perfusion images. In many embodiments, a trained computational model is used to extract temporal features of the scans of perfusion images from the generated time-intensity curves. In several embodiments, a trained computational model is used to extract spatial features from the scans of perfusion images. In some embodiments, a perfusion parametric map is generated utilizing the extracted temporal features and the extracted spatial features.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 provides a flow diagram of a method to generate parametric perfusion maps in accordance with various embodiments.

FIG. 2 provides a diagram of perfusion image acquisition utilizing a bolus contrast and CT imager in accordance with various embodiments.

FIG. 3 provides acquired scans of perfusion images acquired over time in accordance with various embodiments.

FIG. 4 provides an image of generating time-intensity curves of voxels utilizing preprocessing procedures in accordance with various embodiments.

FIGS. 5A and 5B each provide a conceptual illustration of an exemplary convolutional neural network with a time encoder and spatial encoder, which can be utilized in accordance with various embodiments.

FIG. 6 provides a conceptual illustration of a computational processing system in accordance with various embodiments.

FIG. 7 provides a comparison of TMAX perfusion parametric maps of a stroke patient from CT acquired images generated utilizing RAPID software and a trained computational model in accordance with various embodiments.

FIG. 8 provides an example of poor selection of AIF and VOF by the RAPID software of a stroke patient due to movement during CT imaging.

FIG. 9 provides a comparison of TMAX perfusion parametric maps of the stroke patient of FIG. 8, which were generated utilizing RAPID software and a trained computational model in accordance with various embodiments.

FIG. 10 provides a comparison of TMAX perfusion parametric maps of a stroke patient from MR acquired images generated utilizing RAPID software and a trained computational model in accordance with various embodiments.

FIG. 11 provides an example of poor selection of AIF and VOF by the RAPID software of a stroke patient during MR imaging.

FIG. 12 provides a comparison of TMAX perfusion parametric maps of the stroke patient of FIG. 11, which were generated utilizing RAPID software and a trained computational model in accordance with various embodiments.

### DETAILED DESCRIPTION

Turning now to the drawings and data, various methods and systems for generating perfusion maps utilizing deep learning computational models are described, in accordance with various embodiments. In several embodiments, a perfusion map is generated for the diagnosis of a cerebrovascular disorder. In many embodiments, one or more perfusion maps of cerebral blood volume (CBV), cerebral blood flow (CBF), mean transit time (MTT), and time to max (TMAX) are generated, which can be useful in generating diagnostics in a number of cerebrovascular disorders. For example, in some embodiments, the infarct core and penumbra of a stroke are determined utilizing one or more of the computed perfusion maps. Based on results, and in accordance with some embodiments, one or more of the computed perfusion maps is utilized to determine a treatment plan.

A goal in clinical assessment utilizing medical imaging, such as computed tomography (CT) perfusion or magnetic resonance (MR) perfusion images, is to provide quantitative perfusion parametric maps useful for proper diagnosis. Perfusion imaging is a widely applied technique for the evaluation of acute ischemic stroke patients, including prediction of tissue at risk and clinical outcome; it is also used in the assessment of brain tumors, metastases, and other cerebrovascular diseases, and it is often useful in the diagnosis of seizures and pathogenic infections.

In accordance with various prior methodologies, different mathematical algorithms have been used to generate these perfusion parametric maps, which typically requires the selection of the arterial input function (AIF) and venous output function (VOF) regions within the medical images. Multiple commercial or free software for CT or MR perfusion analysis are available, in which the selection of the AIF/VOF is made either manually or automatically, however, poor selection of the AIF/VOF results in inaccurate perfusion maps incapable of providing a proper diagnosis. Manual AIF/VOF selection is time consuming and influenced by operator preference, while automated selection methods can lack robustness when images capture patient motion and/or contain noise. Substantial variations in the perfusion parametric maps due to the selection of AIF/VOF have been reported. Accordingly, it would be an advance in the art to provide a standardized computational method that obviates the need for selection of an AIF and/or of a VOF.

Generally, in accordance with various currently practiced methodologies to generate a perfusion map, a time-intensity curve (C(t)) is generated at voxels of the selected AIF and/or the selected VOF by subtraction of unenhanced baseline images at each time point. The residual function, R(t), represents the fraction of contrast agent that remains in the voxel at a time t after its arrival. R(t) is then calculated or derived by deconvolving the C(t) with the selected AIF. Perfusion parameters of each (CBF, MTT, TMAX, etc.) are derived from the features of the waveforms of R(t), VOF(t), and AIF(t), such as peak amplitude, width of waveform, time-to-peak, area-under-the-curve, etc. Mathematically, the AIF is used to reduce the variability of the perfusion parameters (CBV, CBF, MTT, and TMAX) caused by inter-patient differences, such as injection protocol, catheter size, cardiac output, amount and concentration of the bolus, etc. Mathematically, the VOF is used as a scaling factor that improves the absolute quantitative accuracy of the CBV and CBF estimation. The AIF is often placed at the proximal segments (A1, A2) of the anterior cerebral artery or proximal segment (M1) of the middle cerebral artery. The VOF is often placed at a large dural venous sinus, such as transverse sinus, torcula, straight sinus, or distal superior sagittal sinus.

In the present disclosure, in accordance with several embodiments, perfusion parametric maps are generated by deep learning computational methods using an ensemble of neural networks. In many embodiments, AIF and VOF are not explicitly determined, and instead the waveforms of a plurality of the voxels are collectively assessed simultaneously to generate perfusion parametric maps (e.g., CBV, CBF, MTT, and TMAX). In some embodiments, in order to train the deep learning model, perfusion parametric maps generated by an FDA cerebrovascular imaging software RAPID (iSchemaView Co.) were used.

In some embodiments, perfusion parametric maps are generated utilizing a model with a number of components. In some embodiments, the model includes a component that is trained to reduce artifacts in the images (such as motion, scanner noise, etc.). In some embodiments, a neural network is trained and utilized to reduce image artifacts. In many embodiments, the cleaned images are then preprocessed before passing through further components. In various embodiments, one or more of the following preprocessing steps are employed: skull-stripping (extracting the brain parenchyma), co-registration of each slice across different time points (to remove motion artifact), intensity normalization (scaling the voxel intensity to a range between 0 and 1), interpolation to generate a uniformly sampled waveform (as CTP datasets may have variable sampling rates) and inverting signal of images (which may be useful in MR generated images). In some embodiments, explicit inversion of signal is not performed, even for MR generated images. In some embodiments, the model includes a component that involves deep learning of the temporal information embedded in the dataset. To extract temporal features, in some embodiments, convolutional filters are employed to extract the features from density waveforms of each pixel analyzed. In some embodiments, the model includes a component that involves deep learning of the spatial distribution of the dataset. To extract spatial features, in some embodiments, a modified U-Net architecture with convolutional filters is employed to extract spatial information embedded in each medical image slice. As should be understood, a model can use one, two, or all of the components, or any other component useful for computing a perfusion parametric map.

In some embodiments, a computational system is provided to implement the one or more components. Such a system can include any combination of hardware and/or software. For example, it can include computer systems that transmit and receive medical imaging data via a network. Real-time software processes can parse medical image header information to identify prespecified commands for medical image processing and can execute those commands on the receiving computer without user interaction. Processed medical images can be automatically tagged by setting the image header and sent to display devices, such as picture archiving and communication systems (PACS).

### Generating Perfusion Parametric Maps

Several embodiments are directed to generating perfusion parametric maps. In many embodiments, a perfusion parametric map is generated without selection of an AIF. In many embodiments, a perfusion parametric map is generated without selection of an VOF. In several embodiments, perfusion medical images acquired undergo noise reduction. In many embodiments, time-intensity curves are generated for a one or more voxels of the perfusion images. In some embodiments, preprocessing of the perfusion medical images is performed. In several embodiments, a time decoder process is performed to extract features from the one or more generated time-intensity curves. In many embodiments, a process is performed to extract spatial features utilizing the perfusion medical images. In several embodiments, a perfusion parametric map (e.g., CBV, CBF, MTT, and TMAX) is generated by combining extracted temporal features and the extracted spatial features.

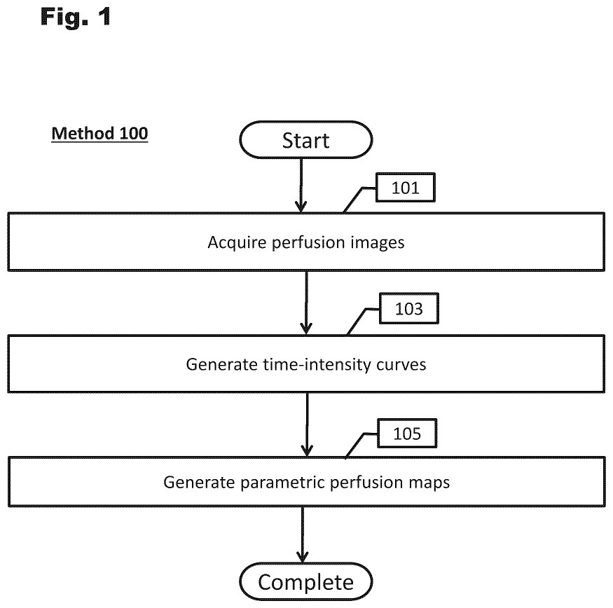

Provided in FIG. 1 is a method to generate a perfusion parametric map in accordance with various embodiments. Process 100 begins with acquiring (101) perfusion images. Perfusion images can be generated in any method in which perfusion (i.e., flow of bodily fluid through an organ or tissue) is monitored. Generally, a contrast agent is utilized and medical images are acquired over a time period. Any contrasting agent and compatible imager can be utilized in accordance with various embodiments. In some embodiments, a bolus of iodinated fluid is injected into circulation to provide contrast. Images can be acquired by any medical imaging modality. Examples of medical imaging modalities include (but are not limited to) magnetic resonance imaging (MRI), X-ray, fluoroscopic imaging, computed tomography (CT), ultrasound sonography (US), and positron emission tomography (PET). Various imaging modalities can be combined, such as PET-CT scanning. Likewise, various image data derived from multiple modalities can be collected and be utilized as training data. In many embodiments, scans of images at multiple planes are acquired, where each scan provides a three-dimensional perspective of perfusion at a particular time point. In many embodiments, several scans are acquired over time, the total set of scans reflecting the perfusion response over time.

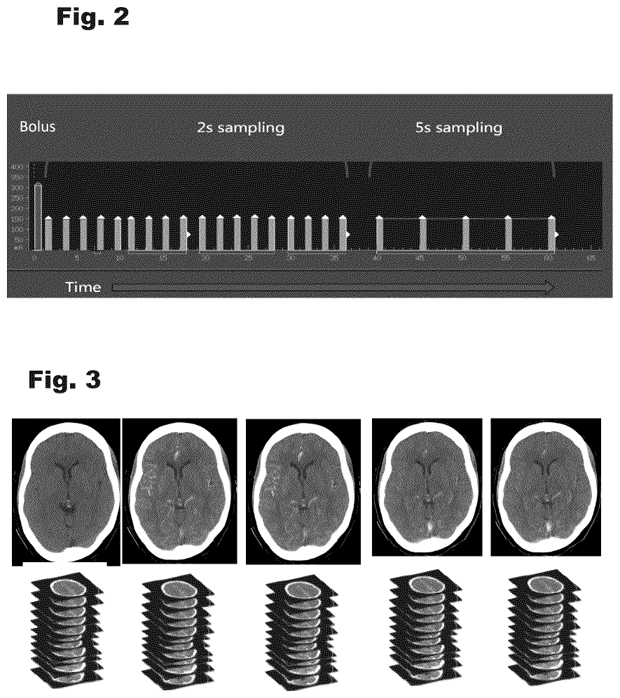

Provided in FIG. 2 is an example of perfusion image acquisition for stroke using iodinated contrast and CT imaging. In this example, a scan of several images in a number of axial planes are acquired for each timepoint. For CT imaging, a scan can be generated every 1 to 2 seconds for the arterial phase, which is typically about 30 to 45 seconds after bolus contrast injection; then scans are acquired every 2 to 5 seconds for the venous phase, which is after the arterial phase and up to 60 to 75 seconds after bolus contrast injection. For MR imaging, a scan can be generated every 1 to 2 seconds for the arterial phase and the venous phase, as radiation is less of an issue with this imaging modality. The precise timing of scan acquisition, however, is largely dependent on the imaging machinery utilized and user preference and thus can be adjusted accordingly. This repeated scanning results in acquisition of a four-dimensional time resolved perfusion data (see an example in FIG. 3).

Often the acquired perfusion images have some associated noise. In some instances, noise may arise during image acquisition, such as (for example) noise generated due to movement of the patient, which is a common occurrence, especially with patients that are suffering from a stroke. Other sources may also lead to noise within the acquired images. Accordingly, in some embodiments, noise is reduced in one or more of the acquired images. Any appropriate means to reduce noise within acquired images can be utilized. In some embodiments, a denoising autoencoder and autodecoder is used to denoise the acquired images.

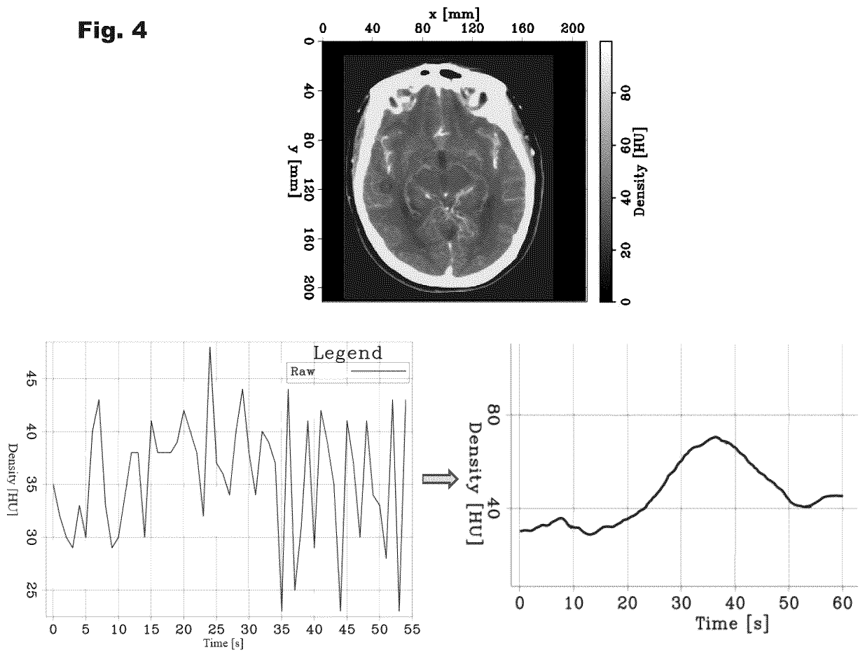

As depicted in FIG. 1, method 100 generates (103) time-intensity curves utilizing one or more voxels. Any voxel or region comprising a plurality of voxels may be utilized to generate time-intensity curves. In several embodiments, voxels for generating curves are not voxels associated with a selected AIF or the VOF. In many embodiments, time-intensity curves are generated from all the voxels of the brain. In some embodiments, time-intensity curves extracted from all the brain tissue voxels are compared and analyzed collectively alongside the time-intensity curves from voxels belonging to arteries and veins, without explicit pre-determination of a representative artery for AIF or a representative vein for VOF. In many embodiments, prior to generating time-intensity curves, one or more preprocessing procedures is performed. Preprocessing procedures include (but are not limited to) co-registering the images at different time-points (to better align the scans), spatial filtering (to remove spatial noise), resample and interpolate the data set (to standardize the number of time points), and skull stripping (removing voxels of skull bone in cerebral images). In some embodiments, unlike conventional methods for analyzing contrast in MR scans, the signal is not inverted prior to generating time intensity curves and utilizing within the computational model to generate perfusion parametric maps. FIG. 4 provides an example of generated time-intensity curves. As can be seen, preprocessing of the image data provides a smoothed curve.

Method 100 further generates (105) parametric perfusion maps. Parametric maps that can be generated include (but are not limited to) cerebral blood volume (CBV), cerebral blood flow (CBF), mean transit time (MTT), and/or time to max (TMAX). For CT imaging, it is common generate absolute CBV and CBF maps. For MR imaging, on the other hand, it is common to generate relative CBV and CBF maps, comparing the injured side of the brain with the healthy side. It should be understood, however, that any imaging modality can be utilized in the generation of absolute parametric maps or relative parametric maps. To generate parametric perfusion maps, in accordance with various embodiments, a trained computational learning model can be utilized to extract temporal data and/or spatial data derived from the acquired images. In some embodiments, temporal data is extracted from the time-intensity curves. For instance, in some embodiments, a time-encoder model is utilized to extract features from the time-intensity curves. Temporal features that can be extracted include (but are not limited to) delayed travel time, time to fill tissue, and rate at which tissue is being filled. Similarly, in some embodiments, spatial data is extracted from the acquired images. For instance, in some embodiments, a spatial-encoder model is utilized to extract features from the acquired images.

Any appropriate computational learning model or combination of learning models that can handle spatial data and sequential (time-series) data can be utilized, including (but not limited to) deep neural networks (DNN), convolutional neural networks (CNN), recurrent neural networks, long short-term memory (LSTM) networks, kernel ridge regression (KRR), and/or gradient-boosted random forest decision trees. Likewise, any appropriate model architecture can be utilized that provides an ability to predict disorder progression. In some embodiments, no supervision is provided to train the model. In some embodiments, attention gates are utilized to focus the model to train specific target structures within the collected images. To train the model, in accordance with various embodiments, established and accurate parametric maps can be utilized. In some embodiments, parametric maps generated by the FDA-approved cerebrovascular imaging software RAPID (iSchemaView, Inc., Menlo Park, CA).

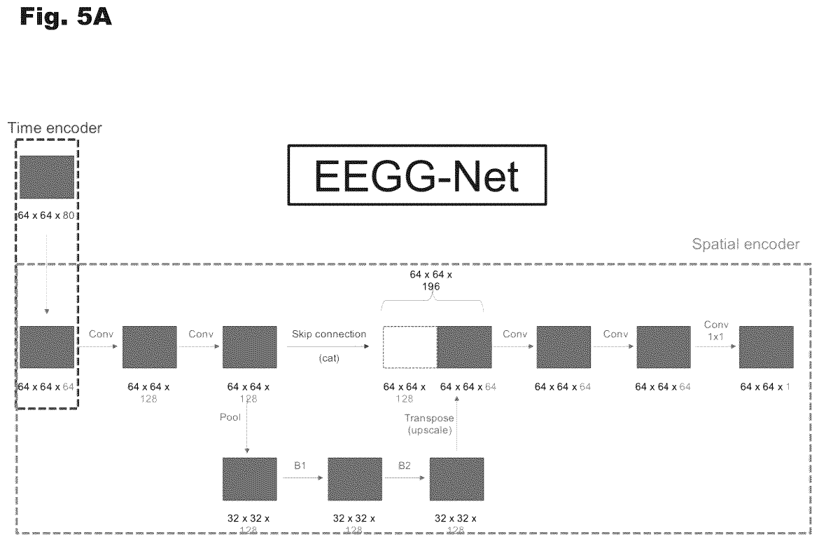

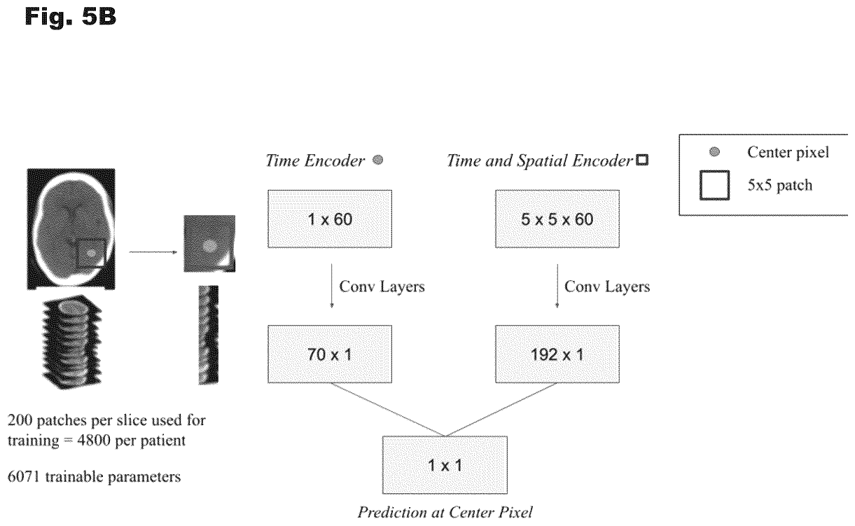

Provided in FIG. 5A is an example of convolutional neural network with a time encoder and spatial encoder to extract features. In this particular example, the input to the model is 256×256×80 data set, representing 80 total timepoint scans of 256×256 acquired images.

Provided in FIG. 5B is another example of a convolutional neural network with a time encoder and time and spatial encoder to extract features. In this example, the scans are split into 5×5 patches, each patch having a central pixel. The central pixel for 60 timepoints (1×60) is utilized as the input within the time encoder. Similarly, 5×5patches for 60 timepoints (5×5×60) is utilized for input in the time and spatial encoder. In this particular example, training is performed on a subset of patches (e.g., 200 patches) and prediction is performed on all overlapping patches such that each pixel is assessed. The time encoder and time and spatial encoder are combined to provide a prediction at each central pixel.

In several embodiments, the extracted temporal and spatial data is utilized to calculate a parameter for each voxel assessed. To calculate a parameter, in accordance with many embodiments, the computational model applies filters to the input image to extract features embedded in the images. Each filter can have a variable size and can have a weight and performs a mathematical operation on its input. By adjusting the weights of the various filters, different features can be extracted. Some filters can be designed to extract spatial features from the input images, and some filters can be designed to extract temporal features from the time-intensity curves extracted from the input images. In many embodiments, the computational model applies many filters both in parallel and in stacks (one layer after another layer). The stacking of filters allows a hierarchical decomposition of the input images into more complex and abstract features. In several embodiments, the computational model utilizes the spatial features and temporal features to determine the output (perfusion parameter) for each voxel. Weights of the filter are adjusted to optimize the perfusion parameter for each voxel. In some embodiments, a TMAX is calculated for each voxel. In some embodiments, a CBV is calculated for each voxel. In some embodiments, a CBF is calculated for each voxel. In some embodiments, a MTT is calculated for each voxel. Based on computed parameters, diagnoses can be performed. For instance, in parametric maps of stroke lesions, the infarct core and penumbra can be defined. Typically, the infarct core is defined by having less than 30% CBF. The infarct core can also be defined by having less than normal CBV and much greater than normal TMAX. The penumbra is often defined by having a TMAX greater than normal (e.g., TMAX>6 s). The penumbra can also be defined by having a less than normal CBF and a greater than normal CBV.

Furthermore, diagnostic parametric maps can inform a medical professional how to treat an individual. For example, parametric maps of a stroke lesion can inform where to perform a thrombectomy based on lesion location and severity. Generated parametric maps can be used to determine which (and how much) tissue is rescuable, by calculating mismatch volume and mismatch ratio, as is understood by professionals in the field.

While specific examples of processes for generating parametric perfusion maps utilizing perfusion images are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for generating parametric perfusion maps appropriate to the requirements of a given application can be utilized in accordance with the various embodiments.

### Computational Processing System

A computational processing system to generate parametric perfusion maps in accordance with various embodiments of the disclosure typically utilizes a processing system including one or more of a CPU, GPU and/or neural processing engine. In a number of embodiments, captured image data is processed using an Image Signal Processor and then the acquired image data is analyzed using one or more machine learning models implemented using a CPU, a GPU and/or a neural processing engine. In some embodiments, the computational processing system is housed within a computing device directly associated with the imaging device. In some embodiments, the computational processing system is housed independently of the imaging device and receives the acquired images. In certain embodiments, the computational processing system is in communication with the imaging device. In various embodiments, the computational processing system communicates with the imaging device by any appropriate means (e.g., a wireless connection). In certain embodiments, the computational processing system is implemented as a software application on a computing device such as (but not limited to) mobile phone, a tablet computer, a wearable device (e.g., watch and/or AR glasses), and/or portable computer.

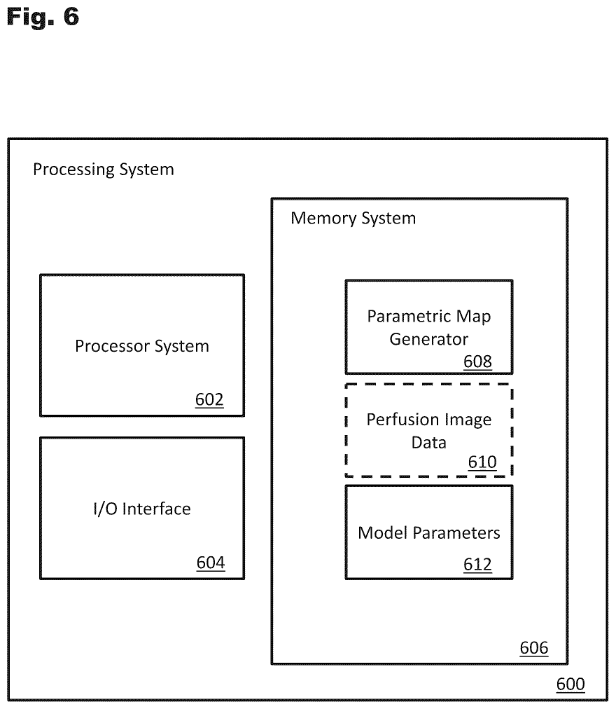

A computational processing system in accordance with various embodiments of the disclosure is illustrated in FIG. 6. The computational processing system 600 includes a processor system 602, an I/O interface 604, and a memory system 606. As can readily be appreciated, the processor system 602, I/O interface 604, and memory system 606 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a parametric map generator application 608. The parametric map generator application can be downloaded and/or stored in non-volatile memory. When executed, the parametric map generator application is capable of configuring the processing system to implement computational processes including (but not limited to) the computational processes described above and/or combinations and/or modified versions of the computational processes described above. In several embodiments, the parametric map generator application 608 utilizes perfusion image data 610, which can be stored in the memory system, to perform image processing including (but not limited to) reducing image noise, performing preprocessing procedures, and generating time-intensity curves. In certain embodiments, the parametric map generator application 608 utilizes model parameters 612 stored in memory to process acquired image data using machine learning models to perform processes including (but not limited to) extracting temporal features and extracting spatial features. Model parameters 612 for any of a variety of machine learning models including (but not limited to) the various machine learning models described above can be utilized by the parametric map generator application. In several embodiments, the perfusion image data 610 is temporarily stored in the memory system during processing and/or saved for use in training/retraining of model parameters.

While specific computational processing systems are described above with reference to FIG. 6, it should be readily appreciated that computational processes and/or other processes utilized in the provision of parametric map generation in accordance with various embodiments of the disclosure can be implemented on any of a variety of processing devices including combinations of processing devices. Accordingly, computational devices in accordance with embodiments of the disclosure should be understood as not limited to specific imaging systems, computational processing systems, and/or parametric map generator systems. Computational devices can be implemented using any of the combinations of systems described herein and/or modified versions of the systems described herein to perform the processes, combinations of processes, and/or modified versions of the processes described herein.

### EXEMPLARY EMBODIMENTS

The embodiments of the invention will be better understood with the various examples described herein. The examples provided here compare standard practices of generating parametric perfusion maps with the deep learning methods of the current disclosure.

### Example 1

### Comparison of Generating CT Perfusion Parametric Maps of a Patient Utilizing a Trained Computational Model and a Prior Art Method Utilizing RAPID Software to Select AIF and VOF

In this example, a trained computational model was developed to utilize acquired perfusion images of stroke patients to generate perfusion maps from CT scans. The perfusion maps generated by the trained computational model was compared with a prior art method that utilizes RAPID software to select AIF and VOF to generate perfusion maps. Using the example architecture shown in FIG. 5A, the trained computational model utilizes perfusion images acquired over a period a time, and denoises these images. The images are utilized to generate time-intensity curves from each of the voxels of the perfusion within the brain scan images. A TMAX parametric perfusion map is generated utilizing the computational model to extract temporal features from the generated time-intensity curves and spatial features from the acquired images. The computational model was trained utilizing successfully generated TMAX parametric perfusion maps that were generated by the prior art method utilizing RAPID software to select AIF and VOF.

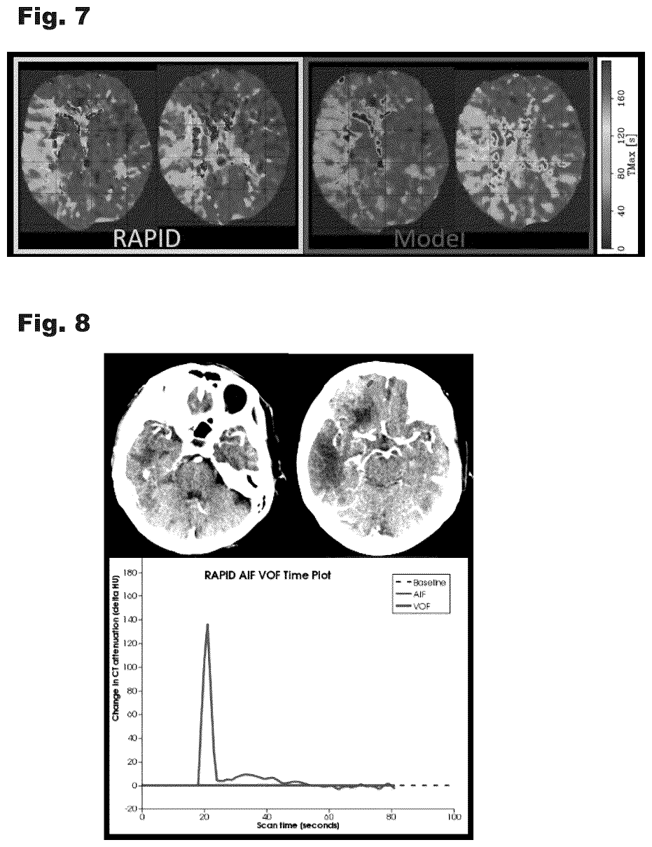

The computational model generated TMAX parametric perfusion maps of the same caliber of the prior art method utilizing RAPID software to select AIF and VOF (FIG. 7). These results demonstrate that a trained computational model can generate high-quality parametric perfusion maps from CT scans that are useful for diagnosing and treating stroke.

### Example 2

### Failures of RAPID Software to Select AIF and VOF in Generation of CT Perfusion Parametric Maps

In this example, the RAPID software does not accurately select AIF and VOF due to motion of a stroke patient during CT imaging. This results in a TMAX parametric perfusion map that is not able to properly diagnose the patient. Use of the trained computational model as described in Example 1, on the other hand, is capable of generating a TMAX parametric perfusion map that is able to properly diagnose the patient.

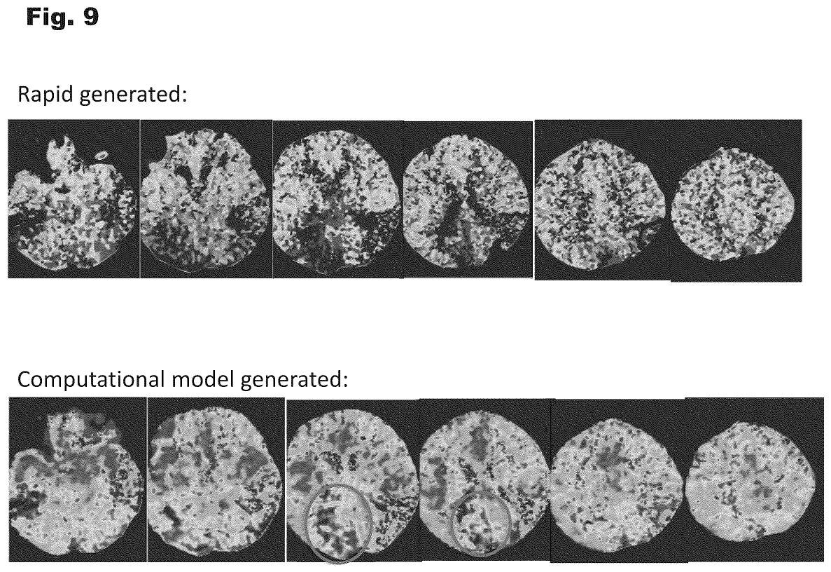

FIG. 8 provides the location of the AIF and VOF selections of the RAPID software in the stroke patient. The RAPID software incorrectly selects the AIF voxel (as indicated by the dot) in a location that is not an artery, resulting in a time-intensity curve that is incorrect. Unlike the sharp peak that is shown in FIG. 8, an accurate AIF time-intensity curve should be a smoother and more rounded rise and fall. Further, the RAPID software could not find the VOF voxel and thus did not even generate a VOF time-intensity curve. The generated TMAX parametric perfusion map results in signal that is unable to identify the infarct and penumbra (FIG. 9; the dark red signifies infarct area, which is spread throughout the generated map). The trained computational model, on the other hand, was capable of generating TMAX parametric perfusion maps capable of accurately identifying the infarct area, the penumbra, and thus the severity of the stroke and guidance on treatment options (FIG. 9; the infarct is identified within the circle).

### Example 3

### Comparison of Generating MR Perfusion Parametric Maps of a Patient Utilizing a Trained Computational Model and a Prior Art Method Utilizing RAPID Software to Select AIF and VOF

In this example, a trained computational model was developed to utilize acquired perfusion images of stroke patients to generate perfusion maps from MR scans. The perfusion maps generated by the trained computational model was compared with a prior art method that utilizes RAPID software to select AIF and VOF to generate perfusion maps. Using the architecture shown in FIG. 5B, the trained computational model utilizes perfusion images acquired over a period a time, and denoises these images. The images are utilized to generate time-intensity curves from each of the voxels of the perfusion within the brain scan images. A TMAX parametric perfusion map is generated utilizing the computational model to extract temporal features from the generated time-intensity curves and spatial features from the acquired images. The computational model was trained utilizing successfully generated TMAX parametric perfusion maps that were generated by the prior art method utilizing RAPID software to select AIF and VOF.

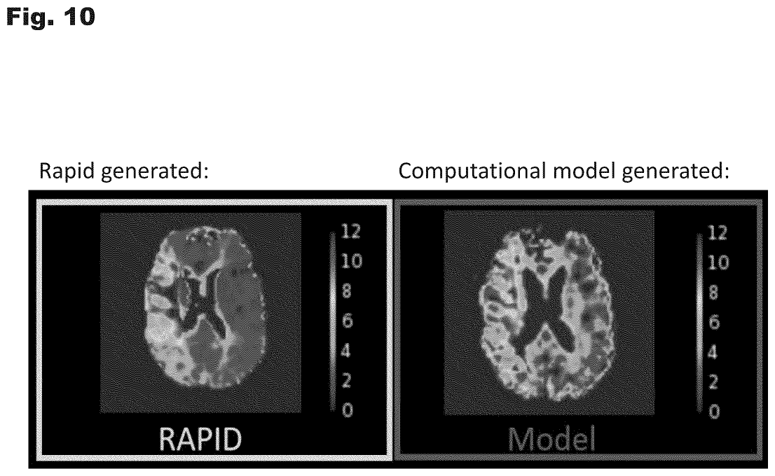

The computational model generated TMAX parametric perfusion maps of the same caliber of the prior art method utilizing RAPID software to select AIF and VOF (FIG. 10). These results demonstrate that a trained computational model can generate high-quality parametric perfusion maps from MR scans that are useful for diagnosing and treating stroke.

### Example 4

### Failures of RAPID Software to Select AIF and VOF in Generation of MR Perfusion Parametric Maps

In this example, the RAPID software suboptimally selects AIF and VOF. A distal branch of the posterior cerebral artery was selected as AIF and the VOF was indeterminate. This results in a TMAX parametric perfusion map that is not able to properly diagnose the stroke. Use of the trained computational model as described in Example 3, on the other hand, is capable of generating a TMAX parametric perfusion map that is able to properly diagnose the stroke.

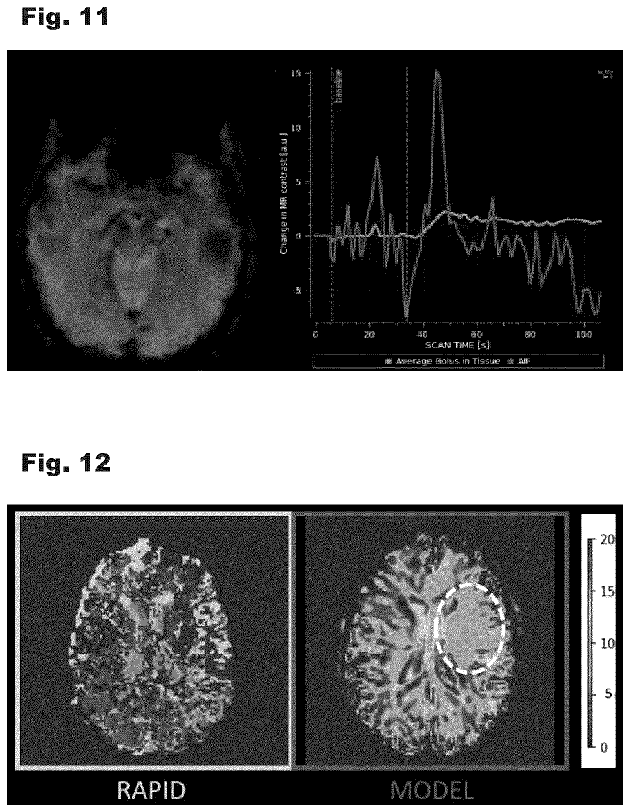

FIG. 11 provides the location of the AIF and VOF selections of the RAPID software in the stroke patient. The RAPID software selected a distal branch of the posterior cerebral artery as the AIF and the VOF was indeterminate. This results in generation of a noisy AIF curve and because the RAPID software could not find the VOF voxel, a VOF time-intensity curve was not generate. The generated TMAX parametric perfusion map results in signal that is unable to identify the infarct and penumbra (FIG. 12; the dark red signifies infarct area, which is spread throughout the generated map). The trained computational model, on the other hand, was capable of generating TMAX parametric perfusion maps capable of accurately identifying the infarct area, the penumbra, and thus the severity of the stroke and guidance on treatment options (FIG. 12; the infarct is identified within the circle).

1. A method of generating a perfusion parametric map, comprising:
providing scans of perfusion images that were acquired over a period of time;
selecting a plurality of voxels of the scans of perfusion images;
for each selected voxel, generating time-intensity curves that reflect the perfusion of a liquid within the scans of perfusion images;
extracting, utilizing one or more trained computational models, temporal features of the scans of perfusion images from the generated time-intensity curves;
extracting, utilizing the one or more trained computational models, spatial features from the scans of perfusion images; and
generating a perfusion parametric map utilizing the extracted temporal features and the extracted spatial features.

2. The method as in claim 1 further comprising denoising the scans of perfusion images.

3. The method as in claim 1 further comprising preprocessing the scans of perfusion images prior to generating the time-intensity curves.

4. The method as in claim 1, wherein the one or more trained computational models incorporate a deep learning model that is capable of handling spatial data and sequential (time-series) data.

5. The method as in claim 1, wherein the one or more trained computational models incorporate a deep neural network (DNN), a convolutional neural network (CNN), a recurrent neural network, a long short-term memory (LSTM) network, a kernel ridge regression (KRR), or a gradient-boosted random forest technique.

6. The method as in claim 1, wherein the perfusion parametric map is a map of: cerebral blood volume (CBV), cerebral blood flow (CBF), mean transit time (MTT), or time to max (TMAX).

7. The method as in claim 1, wherein the perfusion parametric map is utilized to assess perfusion in a cerebral perfusion abnormality.

8. The method of claim 7, wherein the cerebral perfusion abnormality is a cerebrovascular disorder, a hypo-perfusion disorder, or a hyper-perfusion disorder.

9. The method of claim 8, wherein the cerebrovascular disorder is a stroke, a vascular malformation, or a transient-ischemic attack.

10. The method of claim 8, wherein the hypo-perfusion disorder is a post-ictal phase of a seizure or a post-radiation necrosis.

11. The method of claim 8, wherein the hyper-perfusion disorder is a brain malignancy, an ictal phase of a seizure, inflammation, or an infection.

12. The method as in claim 1, wherein the selected voxels do not require explicit determination of arteries and veins.

13. The method as in claim 1, wherein the selected voxels do not require an explicit selection of voxels of an arterial input function and a venous output function.

14. The method as in claim 1, wherein the perfusion parametric map is utilized to assess perfusion in a cerebral perfusion abnormality; and wherein the selected voxels are all the voxels of a brain included in the perfusion images.

15. The method as in claim 1, wherein the scans of perfusion images are captured by at least one of: magnetic resonance imaging (MRI), X-ray, fluoroscopic imaging, computed tomography (CT), ultrasound sonography (US), or positron emission tomography (PET).

16. A computational system for generating a perfusion parametric map, comprising:
a processor system and a memory system, wherein the memory system comprises a parametric map generator application that comprises one or more trained computational model for extracting features;
wherein the parametric map generator application is capable of instructing the processor to:
receive scans of perfusion images that were acquired over a period of time;
select a plurality of voxels of the scans of perfusion images;
for each selected voxel, generate time-intensity curves that reflect the perfusion of a liquid within the scans of perfusion images;
extract, utilizing the one or more trained computational models, temporal features of the scans of perfusion images from the generated time-intensity curves;
extract, utilizing the one or more trained computational models, spatial features from the scans of perfusion images; and
generate a perfusion parametric map utilizing the extracted temporal features and the extracted spatial features.

17. The computational system of claim 16 further comprising an imaging modality, wherein the imaging modality acquired the scans of perfusion images.

18. The computational system of claim 17, wherein the computational system is housed in a computing device directly associated with the imaging modality.

19. The computational system of claim 17, wherein the computational system is housed in a computing device independently of the imaging modality.

20. The computational system of claim 17, wherein the imaging modality is at least one of: magnetic resonance imaging (MRI), X-ray, fluoroscopic imaging, computed tomography (CT), ultrasound sonography (US), or positron emission tomography (PET).

21.-25. (canceled)
